(19) United States
(12) Patent Application Publication
(10) Pub. No.: US 2025/0170103 A1
(43) Pub. Date: May 29, 2025

(54) 4-(6-OXO-2-(TRIFLUOROMETHYL)-3,6-DIHYDROCHROMENO[7,8-D]IMIDAZOL-8-YL)BENZONITRILE FOR USE IN THE TREATMENT OF DISEASES AND CONDITIONS ASSOCIATED WITH MOVEMENT DISORDERS

(71) Applicant: OnTrack Therapeutics Limited, Kent (GB)

(72) Inventors: Thomas PFEIFFER, London (GB); Alleyn Thomas PLOWRIGHT, Peterborough (GB); Man-Ling HO, London (GB)

(21) Appl. No.: 18/868,743

(22) PCT Filed: May 24, 2023
(86) PCT No.: PCT/GB2023/051356

§ 371 Date: Nov. 23, 2024

(30) Foreign Application Priority Data
May 24, 2022 (GB) 2207557.6
Mar. 23, 2023 (GB) 2304262.5

(51) Int. Cl. A61K 31/4188; A61K 45/06; A61P 25/16

(52) U.S. Cl. CPC A61K 31/4188; A61K 45/06; A61P 25/16

(57) ABSTRACT

The present invention relates to the compound 4-(6-oxo-2-(trifluoromethyl)-3,6-dihydrochromeno[7,8-d]imidazol-8-yl)benzonitrile, also known as CF3CN, for use in the treatment of diseases and conditions associated with movement disorder.

### FIELD OF THE INVENTION

The present invention relates to the compound 4-(6-oxo-2-(trifluoromethyl)-3,6-dihydrochromeno[7,8-d]imidazol-8-yl)benzonitrile, also known as CF3CN, for use in the treatment of diseases and conditions associated with movement disorders.

### BACKGROUND TO THE INVENTION

The compound tropoflavin, also known as 7,8-dihydroxyflavone (7,8-DHF), is a naturally occurring flavone found in Godmania aesculifolio, Tridax procumbent and primula tree leaves. It is known to act as a potent and selective agonist of tropomyosin receptor kinase B (TrkB), which is the main signaling receptor of neurotrophin brain-derived neurotrophic factor (BDNF).

Tropoflavin has been shown to have therapeutic efficacy in several animal models including depression, Alzheimer's disease, cognitive deficits in schizophrenia, Parkinson's disease, Huntington's disease, amyotrophic lateral sclerosis, traumatic brain injury, cerebral ischemia, fragile X syndrome and Rett syndrome.

A derivative of tropoflavin, 4-(6-oxo-2-(trifluoromethyl)-3,6-dihydrochromeno[7,8-d]imidazol-8-yl)benzonitrile, also known as CF₃CN, has been shown to be useful in the treatment of diseases and conditions associated with neurodegenerative dysfunction.

Neurodegenerative diseases are caused by the progressive damage or death of neurons, these are nerve cells in the brain whose primary function is to assist in the memory process. The damage or death of neurons leads to a gradual deterioration of the functions controlled by the affected part of the nervous system.

Neurodegenerative diseases are a group of disorders characterized by changes in normal neuronal functioning, leading, in most cases, to neuronal death. Most of these diseases are associated with severe neuronal loss and the impact of these diseases can be catastrophic for the patient and their carers.

According to the National Institute of Neurological Disorders and Stroke (NINDS), there are more than 600 different types of neurological disorders. For many of these disorders there are no adequate treatments and/or therapies that exist. Such disorders include: Amyotrophic lateral sclerosis (ALS); alcohol induced neurotoxicity; Alzheimer's disease; attention deficit disorder; Batten disease; chemotherapy-related cognitive dysfunction; Creutzfeldt-Jakob disease (CJD); dementia with Lewy bodies disease; Down's syndrome; early onset dementia; epilepsy-related cognitive dysfunction; frontotemporal dementia; HIV dementia; mild cognitive impairment; multiple sclerosis-related cognitive dysfunction; normal pressure hydrocephalus; Parkinson's disease-related cognitive dysfunction; posterior cortical atrophy; primary progressive aphasia; prion disease; progressive supranuclear palsy; Rett syndrome; stroke-related cognitive dysfunction; traumatic brain injury; traumatic spinal cord injury and vascular dementia.

Movement disorders are a group of neurological conditions that cause abnormal increased movements, which may be voluntary or involuntary. Movement disorders can also cause reduced or slow movements, the speed, fluency, quality and ease of body movements can also be affected.

According to the American Association of Neurological Surgeons movement disorders are a group of many neurological conditions that can be caused by dysfunction of the brain, genetic conditions or metabolic disorders. Conditions commonly associated with movement disorders include: ataxia; cerebral palsy; cervical dystonia; chorea; dystonia; epilepsy; functional movement disorder; Huntington's disease; multiple sclerosis; multiple system atrophy; myoclonus; Parkinson's disease; Parkinsonism; progressive supranuclear palsy; restless legs syndrome; tardive dyskinesia; Tourette syndrome; tremor disorders such as essential tremor; and Wilson's disease.

Certain movement disorders are additionally associated with serious cognitive impairment, for example, Parkinson's disease and Huntington's disease. Other movement disorders are associated with mild cognitive deficits such as essential tremor. In addition, some movement disorders are brought about by certain medications, for example tardive drug-induced movement disorders which are caused by some antipsychotics and the antiemetic metoclopramide.

A loss or a decrease in TrkB signalling has been shown to be associated with many neurodegenerative conditions and movement disorders including Alzheimer's disease, Huntington's disease, Parkinson's disease, autism, Rett syndrome, sudden infant death syndrome, retinal ganglion cell loss ageing and traumatic brain injury. As such the identification of compounds which are able to act as agonists of the TrkB receptor are of potential use as therapeutic treatments for diseases and conditions associated with movement disorders.

The compound of the invention, 4-(6-oxo-2-(trifluoromethyl)-3,6-dihydrochromeno[7,8-d]imidazol-8-yl)benzonitrile, has been shown to be useful in the treatment of diseases and conditions associated with movement disorders. As such this compound may have utility in treating these diseases for which there is a high unmet need and a huge burden on the patient and their carers.

The patent application WO2011/156479 describes compounds and methods for selective activation of TrkB which may be useful in neuroprotection, however the compound of the invention is not specifically disclosed.

The patent application WO2020/033604 discloses CF₃CN and a method of preparation of the compound. The application additionally describes methods of preventing or treating a BDNF and TrkB related disease or condition. The compound is tested in an in vivo mouse model of Alzheimer's disease. Results suggested that repeated administration of the compound was able to prevent synaptic loss in the 5XFAD mice.

The present application provides data which demonstrates that CF₃CN is able to improve neuronal survival and reduce α-synuclein in a MPP+ in vitro Parkinson's disease model. It was additionally shown to improve survival of motor neurons, integrity of the neurite network and TDP43 mis-localisation in an in vitro ALS disease model. Such findings indicate that CF₃CN is a useful compound for use in the treatment of diseases and conditions associated with movement disorders.

Diseases marked by uncontrolled or unwanted physical movements are defined as movement disorders. These disorders occur when problems with the patient's nervous system or the muscles it acts on occur.

Signs and symptoms of movement disorders vary depending on the underlying cause. In general, signs and symptoms of movement disorders include problems with physical coordination, trouble walking, episodes of uncontrolled movements (such as during a seizure), muscle weakness, twitching, or muscle spasm.

Treatment of movement disorders is very difficult, in many cases, movement disorders cannot be cured, and the goal of treatment is to minimize symptoms and relieve pain. Some are severe and progressive, impairing a patient's ability to move and speak.

While treatment for movement disorders will depend on the underlying cause options for treatment include drug therapies to control symptoms; physical or occupational therapy to help maintain or restore the ability to control movements; Botulinum toxin injections to help prevent muscle contractions; and deep brain stimulation, which is a surgical treatment option that uses an implant to stimulate the areas of the brain that control movement.

There remains a need in the art for an effective treatment for diseases and conditions associated with movement disorders.

### BRIEF SUMMARY OF THE DISCLOSURE

In accordance with a first aspect of the present invention there is provided CF₃CN for use in the treatment of diseases or conditions associated with movement disorder.

Preferably, the diseases or conditions associated with movement disorder is selected from the group: ataxia; cerebral palsy; cervical dystonia; chorea; dystonia; epilepsy; functional movement disorder; Huntington's disease; multiple sclerosis; multiple system atrophy; myoclonus; Parkinson's disease; Parkinsonism; progressive supranuclear palsy; restless legs syndrome; tardive dyskinesia; Tourette syndrome; tremor disorders such as essential tremor; and Wilson's disease.

In some embodiments the neurodegenerative diseases or conditions is treatment resistant.

In a further embodiment the neurodegenerative diseases or conditions is drug-induced movement disorder. Preferably the drug-induced movement disorder is akathisia; tremor; serotonin syndrome; acute dystonic reaction; neuroleptic malignant syndrome; Parkinsonism; or tardive drug-induced movement disorder.

In a further embodiment the disease or condition associated with movement disorder is additionally associated with cognitive impairment.

Preferably the disease or condition which is additionally associated with cognitive impairment is taken from the group consisting of: Huntington's disease; Parkinson's disease; and essential tremor.

Preferably CF₃CNI is administered with one or more pharmaceutically acceptable excipients.

Preferably CF₃CN is formulated in a dosage form selected from a liquid, a lozenge, a fast-disintegrating tablet, a lyophilized preparation, a film, a spray, an aerosol, a sustained-release tablet or capsule, a modified release, a sustained relief, a tablet, a capsule a cream, an ointment, or a mucoadhesive.

Preferably CF₃CN is administered as a single daily dose. Alternatively, CF₃CN is administered as multiple daily doses. Further still CF₃CN is administered two, three, four or five times per day.

Preferably each dose comprises at least 0.001 mg of CF₃CN. More preferably each dose comprises between about 0.001 mg and about 500 mg CF₃CN. Alternatively, each dose comprises between about 500 mg and about 1000 mg of CF₃CN.

In a further embodiment CF₃CN is administered with one or more additional drug products.

In accordance with a second aspect of the present invention there is provided a method of treating diseases or conditions associated with movement disorders in a subject in need thereof, the method comprising administering to the subject a therapeutically effective amount of CF₃CN.

In human therapeutics, the physician will determine the dosage regimen that is most appropriate according to a preventive or curative treatment and according to the age, weight, stage of the disease and other factors specific to the subject to be treated. The compositions, in other embodiments, should provide a dosage of from about 0.0001 mg to about 70 mg of compound per kilogram of body weight per day. Dosage unit forms are prepared to provide from about 0.01 mg, 0.1 mg or 1 mg to about 500 mg, or about 1000 mg, and in some embodiments from about 10 mg to about 500 mg of the active ingredient or a combination of essential ingredients per dosage unit form.

The amount of active ingredient in the formulations provided herein, which will be effective in the prevention or treatment of a disorder or one or more symptoms thereof, will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject.

Exemplary doses of a formulation include milligram or microgram amounts of the active compound per kilogram of subject (e.g., from about 1 microgram per kilogram to about 50 milligrams per kilogram, from about 10 micrograms per kilogram to about 30 milligrams per kilogram, from about 100 micrograms per kilogram to about 10 milligrams per kilogram, or from about 100 microgram per kilogram to about 5 milligrams per kilogram).

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above-described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, administration of the same formulation provided herein may be repeated and the administrations may be separated by at least 1 day, 2 days, 3 days, 5 days, 10 days, 15 days, 30 days, 45 days, 2 months, 75 days, 3 months, or 6 months.

### BRIEF SUMMARY OF THE DRAWINGS

The present invention is described with reference to the figure listed below:

FIG. 1 details the effect of CF3CN in an in vitro MPP+ model to determine reduction of mitochondrial stress at 4-hours.

FIG. 2 details the effect of CF3CN in an in vitro MPP+ model to determine level of α-synuclein at 48-hours.

FIG. 3 details the effect of CF3CN in an in vitro MPP+ model to determine level of DA neurons at 48-hours.

FIG. 4 details the effect of CF3CN in an in vitro MPP+ model to determine level of neurites at 48-hours.

FIG. 5 details the effect of CF3CN on the number of motor neurons.

FIG. 6 details the effect of CF3CN on the neurite network.

FIG. 7 details the effect of CF3CN on extra-nuclear TDP-43.

### DEFINITIONS

Various definitions are made throughout this document. Most words have the meaning that would be attributed to those words by one skilled in the art. Words specifically defined either below or elsewhere in this document have the meaning provided in the context of the present invention as a whole and as typically understood by those skilled in the art.

“Subject,” “individual” or “patient” is used interchangeably herein and refers to a vertebrate, preferably a mammal. Mammals include, but are not limited to, murines, rodents, simians, humans, farm animals, sport animals and pets.

“Treating” or “treatment” of any disease or disorder refers, in some embodiments, to ameliorating the disease or disorder (i.e., arresting or reducing the development of the disease or at least one of the clinical symptoms thereof). Treatment may also be considered to include preemptive or prophylactic administration to ameliorate, arrest or prevent the development of the disease or at least one of the clinical symptoms. Treatment can also refer to the lessening of the severity and/or the duration of one or more symptoms of a disease or disorder. In a further feature, the treatment rendered has lower potential for long term side effects over multiple years. In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter) or both. In yet other embodiments, “treating” or “treatment” refers to delaying the onset of the disease or disorder.

“Therapeutically effective amount” means the amount of a compound that, when administered to a patient for treating a disease, is sufficient to affect such treatment for the disease. The “therapeutically effective amount” will vary depending on the compound, the disease and its severity and the age, weight, adsorption, distribution, metabolism and excretion etc., of the patient to be treated.

“Vehicle” refers to a diluent, excipient or carrier with which a compound is administered to a subject. In some embodiments, the vehicle is pharmaceutically acceptable.

“Active ingredient” or “Active pharmaceutical ingredient” or “API” refers to the novel mesembrine salt of the invention.

“Neurodegenerative dysfunction” refers to any of the disorders listed by the National Institute of Neurological Disorders and Stroke (NINDS)—see https://www.ninds.nih.gov/health-information/disorders for more information. In particular the neurodegenerative dysfunctions of the invention include but are not limited to Amyotrophic lateral sclerosis (ALS); alcohol induced neurotoxicity; Alzheimer's disease; attention deficit disorder; Batten disease; chemotherapy-related cognitive dysfunction; Creutzfeldt-Jakob disease (CJD); dementia with Lewy bodies disease; Down's syndrome; early onset dementia; epilepsy-related cognitive dysfunction; frontotemporal dementia; HIV dementia; mild cognitive impairment; multiple sclerosis-related cognitive dysfunction; normal pressure hydrocephalus; Parkinson's disease-related cognitive dysfunction; posterior cortical atrophy; primary progressive aphasia; prion disease; progressive supranuclear palsy; Rett syndrome; stroke-related cognitive dysfunction; traumatic brain injury; traumatic spinal cord injury; vascular dementia; frontotemporal dementia with motor neuron disease (FTD/MND); multiple system proteinopathy (MSP); frontotemporal lobar degeneration (FTLD); Perry disease; limbic-predominant age related TDP-43 encephalopathy (LATE); cerebral age-related TDP-43 with sclerosis (CART); chronic traumatic encephalopathy (CTE); facial onset sensory and motor neuronopathy (FOSMN); argyrophilic grain disease (AGD); corticobasal degeneration (CBD); spinal muscular atrophy (SMA); progressive muscular atrophy (PMA); progressive bulbar palsy (PBP); distal hereditary motor neuropathies (dHMN); Guillain-Barré syndrome; Kennedy's disease; Hirayama disease; post-polio syndrome; flail arm syndrome; flail leg syndrome; segmental lower motor neuron disease; primary lateral sclerosis (PLS); pseudobulbar palsy; hereditary spastic paraplegia (HSP); Brown-Sequard syndrome; PLA2G6-associated neurodegeneration, including: infantile neuroaxonal dystrophy, atypical neuroaxonal dystrophy, adult-onset dystonia-parkinsonism, autosomal recessive early-onset parkinsonism); POLG-associated neurodegeneration, including: Alpers-Huttenlocher syndrome, myocerebrohepatopathy spectrum, myoclonic epilepsy myopathy sensory ataxia, ataxia neuropathy spectrum, and progressive external ophthalmoplegia; Niemann-Pick type C1; Krabbe disease; neurodegeneration with brain iron accumulation disorders; Friedreich ataxia; HIV-associated neurocognitive disorder/HIV-associated dementia; X-linked Adrenoleukodystrophy (X-ALD); cerebellar ataxia; including: autosomal recessive cerebellar ataxias; autosomal dominant cerebellar ataxias (spinocerebellar ataxias), episodic ataxias; X-linked cerebellar ataxias; and mitochondrial ataxias

“Treatment resistant” is defined as the failure of a disease or disorder to respond positively or significantly to treatment.

“Movement disorders” refers to a group of neurological conditions that cause abnormal increased movements, which may be voluntary or involuntary. Movement disorders can also cause reduced or slow movements, the speed, fluency, quality and ease of body movements can also be affected.

“Diseases or conditions associated with movement disorder” refers to any disease or condition that has been diagnosed as having a movement disorder component including but not limited to: ataxia; cerebral palsy; cervical dystonia; chorea; dystonia; epilepsy; functional movement disorder; Huntington's disease; multiple sclerosis; multiple system atrophy; myoclonus; Parkinson's disease; Parkinsonism; progressive supranuclear palsy; restless legs syndrome; tardive dyskinesia; Tourette syndrome; tremor disorders such as essential tremor; and Wilson's disease.

“Sensorimotor deficit” is a reduction in the sensory and motor development in a subject. Motor development is the use and coordination of muscles of the trunk, arms, legs and hands and sensory development from the environment through sight, sounds, smell, taste and hearing are both either halted during the development process of a subject or decline due to a disease or condition.

“Drug-induced movement disorder” is defined as movement disorders that occur after ingestion of particular drugs. Examples of drug-induced movement disorders include: akathisia; tremor; serotonin syndrome; acute dystonic reaction; neuroleptic malignant syndrome; Parkinsonism; or tardive drug-induced movement disorder.

“4-(6-oxo-2-(trifluoromethyl)-3,6-dihydrochromeno[7,8-d]imidazol-8-yl)benzonitrile”, also known as “CF₃CN”, has a SMILES code N #CC1=CC═C(C2=CC(C3=CC═C4C(N═C(C(F)(F)F)N4)=C3O2)=O)C═C1 and the structure defined below:

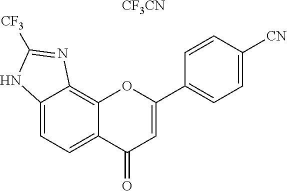 [SMILES: N#Cc1ccc(-c2cc(=O)c3ccc4[nH]c(C(F)(F)F)nc4c3o2)cc1 ; "CF3CN"]

### DETAILED DESCRIPTION OF THE INVENTION

The Example below describes the effect of CF₃CN in two in vitro models of movement disorders and as such demonstrates the compound's ability to treat diseases and conditions associated with movement disorders.

Parkinson's' disease (PD) is a common neurodegenerative movement disorder that affects around 1% of the population over the age of 70 (Martin, 2010). It is the second most common neurodegenerative disease after Alzheimer's disease. The patients suffering PD display symptoms of motor instabilities with resting tremor as the first symptom in 70% of the cases. Other clinical symptoms are rigidity, bradykinesia and postural instability and often include cognitive impairment, depression and sleep disorders (Jankovic, 2008).

Although the pathological mechanisms leading to substantia nigra (SN) degeneration might be multiple, several pathways are considered to be central: protein aggregation linked to proteasomal impairments, mitochondrial dysfunction (also caused by specific DA-toxins), and impairment in dopamine (DA) release. All these pathways are believed to converge and generate reactive oxygen species (ROS) and massive oxidative stress resulting in cell death (Abou-Sleiman et al., 2006).

Dopaminergic neurons-specific toxins (DA-toxins): 1-methyl-4-phenyl-1,2,3,6 tetrahydropyridine (MPTP), 6-hydroxydopamine (6-OHDA) or rotenone, cause parkinsonism in humans (Dauer and Przedborski, 2003). These toxins are largely used by scientists to experimentally mimic the pathology in animals. Any substances reducing DA-toxin neurotoxicity may be useful as a new therapeutic agent for the treatment or prevention of PD.

It has been demonstrated that 24 h after application of MPP+, a non-negligible release of CytC was observed. This late release of CytC induced the moderate caspase-3 activation detected after 48 h of treatment. A significant increase in mitochondrial AIF leakage was also seen, this increase is followed by a significant and significant increase of autophagosomes (a marker of autophagy) in the first hours after application of MPP+. In parallel, aggregation of α-syn was observed inside the neurons. This finding supports the role of the autophagy at the early stage of MPP+ toxicity, to remove the protein aggregates (Vogiatzi et al., 2008). All together these results suggest that at early stage, MPP+ induced autophagy, this process being not able to remove the overwhelming amount of aggregated protein; the neuron therefore, died via necroptosis (Callizot et al., 2019). In addition, PERK pathway which is one of three known main ER stress response is activated in presence of MPP+ (Go et al., 2017).

Example 1 of the present application assesses the neuroprotective effects of the compound CF₃CN in an in vitro model of Parkinson's disease, based on a primary culture of dopaminergic neurons injured with MPP+, a mitochondrial toxin. Early oxidative stress, survival of dopaminergic neurons and aggregation of alpha-synuclein were investigated.

Amyotrophic lateral sclerosis (ALS) is a fatal disorder characterized by subtle onset of focal weakness, typically in the limbs but sometimes in bulbar muscles, which progresses to paralysis of almost all skeletal muscles. There is significant clinico-pathological and genetic overlap between ALS and frontotemporal lobar dementia (FTLD). In ALS, death from respiratory paralysis is typically within five years. The cellular pathology is focal at onset and spreads in a pattern suggesting successive involvement of contiguous neuronal populations. Death of motoneurons occurs in conjunction with deposition of aggregated proteins in motoneurons and oligodendrocytes, and neuroinflammation. While most cases of ALS are sporadic (SALS), about 10% are inherited, usually dominantly (familial ALS, FALS). ALS is designated as an orphan disease, with 1-2/100,000 new cases and a total of ˜5/100,000 total cases each year. In the U.S. and the UK, ALS accounts for about 1/500 to 1/1,000 adult deaths. Strikingly, this implies that approximately 500,000 people now alive in the U.S. will die of ALS. These parameters are largely constant across the globe. The complex pathophysiology of ALS presents many potential therapeutic targets. However, although a wide range of agents has been investigated, only Riluzole (Rilutek®), an antiglutamatergic drug, has demonstrated consistent benefit, and it is the only approved drug for the treatment of the disease. But Riluzole's benefits are modest—it prolongs survival in ALS patients for several months (˜7%) with minimal effect on functional measures. Although the precise molecular pathways that cause the death of motor neurons in ALS remain unknown, some possible mechanisms include a) glutamate-mediated excitotoxicity; b) decrease in neurotrophic factors and their signaling; c) mitochondrial alterations and oxidative damages; d) abnormalities in cytoskeletal proteins resulting in neuronal atrophy and death (Corcia et al. 2007).

TDP-43 (Transactivating response element DNA binding protein 43 kDa) is shown to accumulate in cytoplasm of motor neurons in most cases of ALS. TDP43 is a nuclear RNA-binding protein involved in several aspects of RNA processing that actively shuttles between the nucleus and cytoplasm. In ALS and frontotemporal dementia, TDP43 is excluded from the nucleus, but such cytoplasmic mislocalization is common in neuronal injury or stress, and TDP43-positive inclusions may represent secondary pathology in some neurodegenerative disorders (Davidson et al., 2011).

Genetic rodent models have been used to study ALS pathogenesis, including rats over-expressing human superoxide dismutase 1 (SOD1) with mutations known to cause human familial ALS (eg. SOD1G93A rats). The ALS rat model expressing the mutated form of hSOD-1G93A exhibits features that closely recapitulate the clinical and histopathologic features of the human disease (Nagai et al. 2001). In human or rodent studies (SOD1 models), MN loss is preceded by increased excitability. As increased neuronal excitability correlates with structural changes in dendritic arbors and spines.

Example 2 of the present applications assesses the neuroprotective effects of CF₃CN at various concentrations in an in vitro model of ALS, based on a primary culture of spinal motor neurons, from SOD1 G93A rat embryos, injured with glutamate. Survival of motor neurons, integrity of the neurite network and TDP43 mislocalization were evaluated.

### Example 1: Effects of CF₃CN on Dopaminergic TH-Positive Neurons Injured with MPP+: Mitochondria Stress and Neuronal Survival

Although the pathophysiological processes involved in dopamine (DA) neuron degeneration in Parkinson's disease (PD) are not completely known, apoptotic cell death has been suggested to be involved and can be modelled in dopaminergic cell lines using the mitochondrial toxin 1-methyl-4-phenylpyridinium (MPP+). The aim of the present study was to investigate the potential effect of CF₃CN in an in vitro model of movement disorders.

### Materials and Methods

### Primary Culture of Mesencephalic Neurons

Rat dopaminergic neurons were cultured as described by Visanji et al., 2008 and Callizot et al., 2019. Briefly, pregnant female rat (Wistar) of 15 days of gestation were killed using a deep anesthesia with CO₂ chamber and a cervical dislocation. The midbrains obtained from 15-day-old rat embryos (Janvier, France) were dissected under a microscope. The embryonic midbrains were removed and placed in ice-cold medium of Leibovitz (L15) containing 2% of Penicillin-Streptomycin (PS) and 1% of bovine serum albumin (BSA). The ventral portion of the mesencephalic flexure, a region of the developing brain rich in dopaminergic neurons, was used for the cell preparations.

The midbrains were dissociated by trypsinisation for 20 min at 37° C. (solution at a final concentration of 0.05% trypsin and 0.02% EDTA). The reaction was stopped by the addition of Dulbecco's modified Eagle's medium (DMEM) containing DNAase I grade II (0.5 mg/mL) and 10% of foetal calf serum (FCS). Cells were then mechanically dissociated by 3 passages through a 10 ml pipette. Cells were then centrifuged at 180×g for 10 min at +4° C. on a layer of BSA (3.5%) in L15 medium. The supernatant was discarded, and the cell pellets were re-suspended in a defined culture medium consisting of Neurobasal supplemented with B27 (2%), L-glutamine (2 mM) and 2% of PS solution and 10 ng/mL of Brain-derived neurotrophic factor (BDNF) and 1 ng/mL of Glial-Derived Neurotrophic Factor (GDNF). Viable cells were counted in a Neubauer cytometer using the trypan blue exclusion test. The cells were seeded at a density of 40,000 cells/well in 96 well-plates (pre-coated with poly-L-lysine) and maintained in a humidified incubator at 37° C. in 5% CO2/95% air atmosphere. Half of the medium was changed every 2 days with fresh medium.

For 96 wells plates, only 60 wells were used. The wells of first and last lines and columns were not used (to avoid any edge effect) and were filled with sterile water.

### Mitochondrial Injuries and Application of Compounds

Vehicle: Culture medium (DMSO 0.1%)

Pre-incubation: On day 6 of culture, CF₃CN was solubilized in DMSO, then diluted in the culture medium and pre-incubated with primary neurons for 1 h, before MPP+ exposure. BDNF, solubilized in HBSS, was diluted in the culture medium and pre-incubated with primary neurons for 1 h, before MPP+ exposure. BDNF was used as a validated positive control.

Colour and pH of CF₃CN solutions was determined during solubilization and dilution in the culture medium. pH was assessed using pH paper strips. Stock solution (10 μL, in DMSO) and working solution (100 μL, in culture medium) of CF₃CN was kept for potential further analysis. In case of abnormal colour and/or pH, the batch of CF₃CN was not used.

Injury: One (1) hour after test compound incubation, MPP+ (10 μM) was added to a final concentration of 4 μM, diluted in control medium still in presence of compounds for 4 hours (plate 1) or 48 h (plate 2).

Test compounds were tested on one culture in 96-well plate (n=6 culture wells per condition, 10 experimental conditions per plate) as described in Table 1 below:

TABLE 1
Test compound concentrations

| PLATE 1 (TH/ROS) - 4 hours | PLATE 2 (TH/αSyn) - 48 hours |
|---|---|
| injury | injury |
| Control (vehicle) | Control (vehicle) |
| MPP+ (4 uM, 4 h) / vehicle | MPP+ (4 μM, 48 h)/vehicle |
| +CF₃CN (1 nM) | +CF₃CN (1 nM) |
| +CF₃CN (10 nM) | +CF₃CN (10 nM) |
| +CF₃CN (100 nM) | +CF₃CN (100 nM) |
| +CF₃CN (500 nM) | +CF₃CN (500 nM) |
| +CF₃CN (1 μM) | +CF₃CN (1 μM) |
| +CF₃CN (5 μM) | +CF₃CN (5 μM) |
| +CF₃CN (10 μM) | +CF₃CN (10 μM) |
| +BDNF (50 ng/ml) | +BDNF (50 ng/ml) |

### Immunostaining: TH and Mitochondrial ROS

4 hours after injury, the cell culture supernatant was discarded. Live cells were incubated with MitoSOX™ Red (marker of ROS generated by the mitochondria) for 10 min at 37° C. The MitoSOX™ reagent is cell-penetrant and becomes fluorescent once oxidized by superoxide.

Then, cells were fixed by a solution of 4% paraformaldehyde in PBS, pH=7.3 for 20 min at room temperature. The cells were washed twice in PBS. Cell membranes were permeabilized and non-specific binding sites were blocked with a solution of PBS containing 0.1% of saponin and 1% FCS for 15 min at room temperature.

Then, the cultures were incubated with a monoclonal anti-Tyrosine Hydroxylase (TH) antibody produced in mouse at dilution of 1/10000 in PBS containing 1% FCS, 0.1% saponin, for 2 hours at room temperature. This antibody was revealed with Alexa Fluor 568 goat anti-mouse IgG at the dilution 1/800 in PBS containing 1% FCS, 0.1% saponin, for 1 hour at room temperature.

Nuclei were counterstained with the fluorescent dye Hoechst (sigma, 1/1000): marker of cell number.

### Immunostaining: TH and αSyn

48 hours after intoxication, the cell culture was discarded and the cells were fixed by a solution of 4% paraformaldehyde in PBS, pH=7.3 for 20 min at room temperature. The cells were washed twice in PBS, and then permeabilized. Non-specific sites were blocked with a solution of PBS containing 0.1% of saponin and 1% FCS for 15 min at room temperature. The cultures were incubated with:
a) monoclonal anti-Tyrosine Hydroxylase (TH) antibody produced in mouse at dilution of 1/10000 in PBS containing 1% FCS, 0.1% saponin, for 2 hours at room temperature.
b) polyclonal anti-alpha synuclein (αSyn) antibody produced in rabbit at dilution of 1/200 in PBS containing 1% FCS, 0.1% saponin, for 2 h at room temperature

These antibodies were revealed with Alexa Fluor 488 goat anti-mouse IgG at the dilution 1/800 and with Alexa Fluor 568 goat anti-rabbit IgG at the dilution 1/400 in PBS containing 1% FCS, 0.1% saponin, for 1 h at room temperature.

### Automatic Computer Analysis

For each condition, 20 pictures (representing the whole well area) were automatically taken using ImageXpress® (Molecular Devices) at 10× magnification using the same acquisition parameters. From images, analyses were directly and automatically performed by MetaXpress® (Molecular Devices).

The following read-outs were measured:
Total number of TH neurons (TH positive neurons)
Total neurite network of TH positive neurons (in μm)
Analysis of ROS in TH(+) neurons (overlapping between TH and mitochondrial ROS in μm²).
α-syn aggregation (overlapping between TH and α-syn staining, in μm²)

### Statistical Analysis

All values were expressed as mean+/−SEM (standard error of the mean). Statistical analysis was performed using one-way ANOVA, followed by a Dunnett's or a LSD Fisher's test, using GraphPad Prism. p<0.05 was considered significant.

### Results

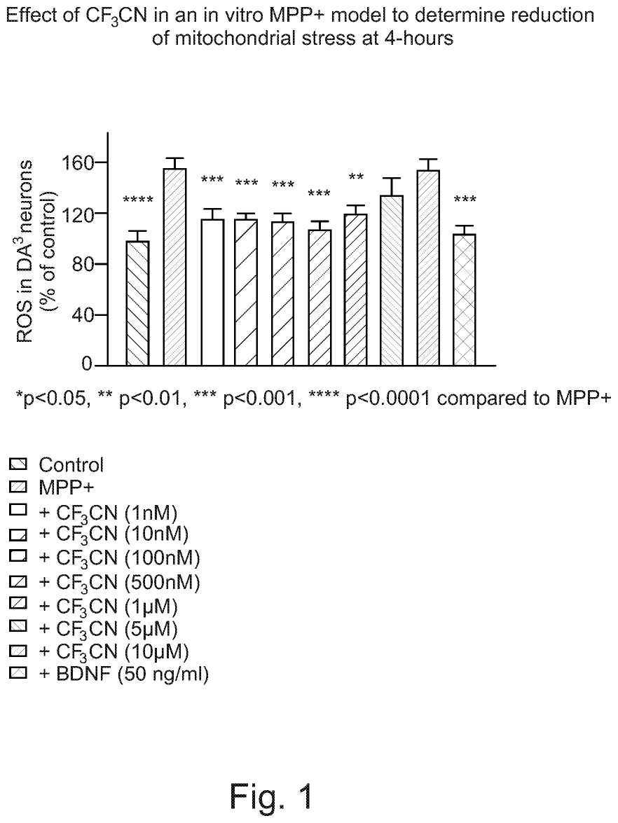

FIG. 1 details the effect of CF₃CN on the reduction of mitochondrial stress after four hours incubation with the test compound, vehicle or the positive control BDNF. As can be seen the test article CF₃CN statistically significantly reduced ROS at concentrations between 10 nm to 1 μm.

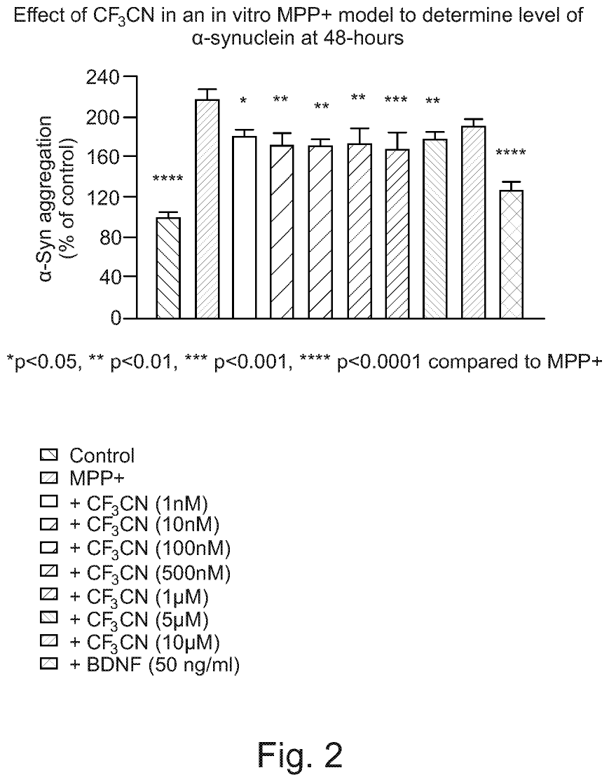

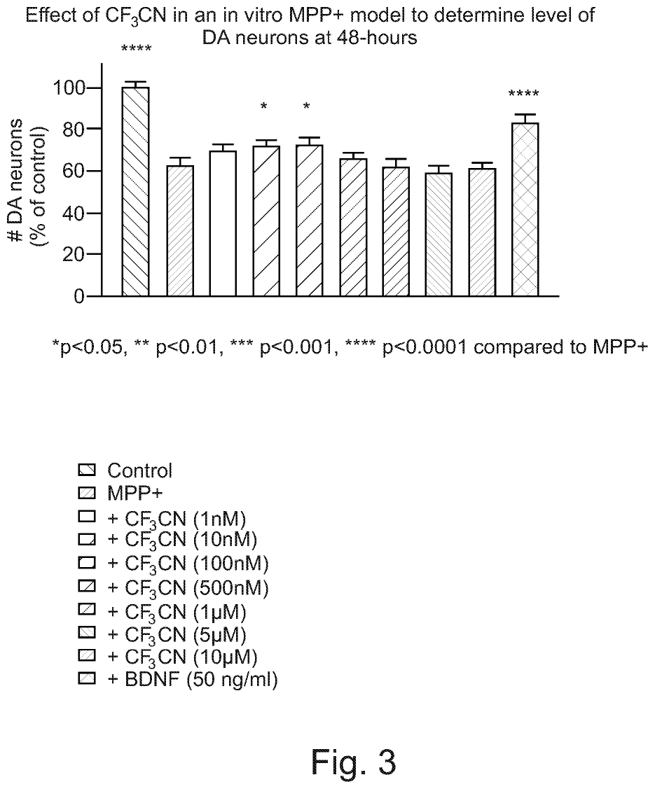

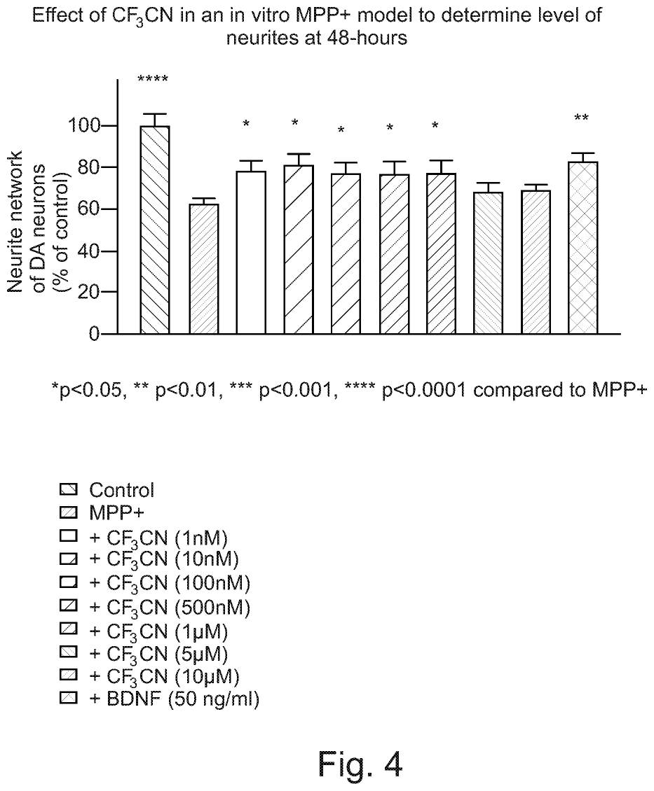

FIGS. 2 to 4 detail the effect of CF₃CN on the levels of biomarkers of Parkinson's disease and dopaminergic cell death after 48 hours incubation. As can be seen CF₃CN was able to statistically significantly reduce the level of α-syn at all dose levels except for 10 μm (FIG. 2). At concentrations of 10 and 100 nm CF₃CN was shown to increase the number of DA neurons (FIG. 3) and at concentrations of 1 nm to 1 μm CF₃CN was shown to increase the level of neurites.

### Conclusion

The ability of CF₃CN to reduce ROS demonstrates that the test compound is able to reduce mitochondrial stress and as such might be a useful compound for use in the treatment of movement disorders.

Furthermore, the ability of CF₃CN to statistically significantly reduce the levels of α-syn and increase the levels of DA neurons and the neurite network demonstrates the ability of this compound to be a useful treatment in movement disorders such as Parkinson's disease.

### Example 2: Effects of CF₃CN on Survival of Motor Neurons, Integrity of the Neurite Network and TDP43 Mislocalization in Primary SOD1 Transgenic Spinal Cord Motor Neurons Following Glutamate Insult

The aim of this study was to assess the neuroprotective effects of CF₃CN at various concentrations in an in vitro model of ALS, based on a primary culture of spinal motor neurons, from SOD1 G93A rat embryos, injured with glutamate. Survival of motor neurons, integrity of the neurite network and TDP43 mislocalization will be evaluated.

### Materials and Methods

### Genotyping of SOD1 Tg Embryos

Female rats (Sprague Dawley, Janvier) were mated with Tg SOD1G93A rats (Taconic Bioscience, US). Pregnant females of 14 days of gestation were dissected. On the day of dissection, a piece of each embryo brain (˜3 mm³) was placed in a 2 mL tube free DNase with a new scalpel. The DNA was extracted with an SYBR Green Extract-N-Amp tissue PCR kit (Sigma Aldrich).

Briefly, 120 μL of extraction solution was put on each piece of embryo heads. Then, they were incubated for 10 min at room temperature. At the end of this incubation period, the heads were incubated for 5 min at 95° C. Immediately after this last incubation, 100 μL of neutralizing solution was added; each DNA extract was diluted at 1/40 and stored at +4° C. until used.

SOD1G93A gene will be determined using genomic fragment with human SOD1 primers (5′-CATCAGCCCTAATCCATCTGA-3′; 5′-CGCGACTAACAATCAAAGTGA-3′). The SOD1 primers were diluted at 3 μM in sterile ultrapure water. Briefly, a mix for PCR was prepared with ultrapure water (4 μL per sample), primer at 3 μM (2 μL per sample) and Master Mix (10 μL per sample). In a PCR 96 wells plate, 16 μL of PCR mix was added in each well. 4 μL of each diluted DNA was added according to a plan deposit. The RT-PCR was run using the CFX96 Biorad RT-PCR system, using the following program: Initial denaturation (95° C., 20 sec)/45 cycles (95° C., 10 sec; 65° C., 10 sec; 72° C., 30 sec)/Melt curve (95° C., 15 sec; 64° C., 1 min; 90° C., 30 sec; 60° C. 15 sec). The amplification plots and melt curves were analyzed with the Biorad software. The results for each sample were compared to negative control (ultrapure water) and to the positive control (DNA from Tg embryos).

### Primary Culture of Spinal Motor Neuron

Rat spinal cord motor neurons (MNs) were cultured as described by Boussicault et al., 2020 and Wang et al. 2013. Briefly, SOD1 Tg fetuses were placed in ice-cold L15 Leibovitz medium with a 2% penicillin (10,000 U/ml) and streptomycin (10 mg/ml) solution (PS) and 1% bovine serum albumin (BSA).

Spinal cords were treated for 20 min at 37° C. with a trypsin-EDTA solution at a final concentration of 0.05% trypsin and 0.02% EDTA. The dissociation was stopped by addition of Dulbecco's modified Eagle's medium (DMEM) with 4.5 g/liter of glucose, containing DNAse I grade II (final concentration 0.5 mg/mL) and 10% fetal calf serum (FCS). Cells were mechanically dissociated by three forced passages through the tip of a 10-ml pipette. Cells were then centrifuged at 515×g for 10 min at 4° C. The supernatant was discarded, and the pellet was resuspended in a defined culture medium consisting of Neurobasal medium with a 2% solution of B27 supplement, 2 mmol/liter of L-glutamine, 2% of PS solution, and 10 ng/mL of brain-derived neurotrophic factor (BDNF). Viable cells were counted in a Neubauer cytometer, using the trypan blue exclusion test. The cells were seeded at a density of 20,000 per well in 96-well plates precoated with poly-L-lysine and will be cultured at 37° C. in an air (95%)-CO₂ (5%) incubator. The medium was changed every 2 days. The wells of the first lines and columns were not used for culture (to avoid any edge effect) and were filled with sterile water. The spinal cord motor neurons were injured with glutamate after 13 days of culture.

### Test Compounds and Glutamate Exposure

Vehicle: Culture medium (DMSO 0.1%)

Pre-incubation: On day 13 of culture, CF₃CN and riluzole were solubilized in DMSO, then diluted in the culture medium and pre-incubated with primary neurons for 1 h, before injury. BDNF, solubilized in HBSS, was diluted in the culture medium and pre-incubated with primary neurons for 1 h, before glutamate insult. Riluzole was used as a validated positive control. BDNF, a classical neurotrophic factor, was used as an endogenous activator of the signalling pathway under investigation.

Color and pH of CF₃CN solutions was determined during solubilization and dilution in the culture medium. pH was assessed using pH paper strips. Stock solution (10 μL, in DMSO) and working solution (100 μL, in culture medium) of CF₃CN was kept for potential further analysis. In case of abnormal color and/or pH, the batch of CF₃CN was not used.

Glutamate injury: On day 13 of culture, glutamate was added to a final concentration of 5 μM diluted in control medium in presence of the compounds for 20 min. After 20 min, glutamate was washed out and fresh culture medium with the test compound, was added for an additional 24 hours.

### Organization of Culture Plates

Test compounds were tested on one culture in 96-well plate (n=6 culture wells per condition as detailed in Table 2 below:

TABLE 2
Concentrations of test compound
PLATE 1 (MAP2/TDP43) - 24 hours injury

Control (vehicle, 0.1% DMSO)
Glutamate (5 μM, 20 min)/vehicle
+CF₃CN (1 nM)
+CF₃CN (10 nM)
+CF₃CN (100 nM)
+CF₃CN (1 μM)
+CF₃CN (5 μM)
+CF₃CN (10 μM)
+Riluzole (5 μM)
+BDNF (50 ng/ml)

All conditions were at 0.1% DMSO. To ensure the 0.1% of DMSO the stock solution of CF₃CN was made at 10 mM in pure DMSO (for a final concentration into well at 10 μM). For Riluzole the stock solution was made at 5 mM in pure DMSO.

### End Point Evaluation

### Immunostaining: MAP-2 and TDP43

24 hours after intoxication, the supernatants were collected and stored at −80° C., and cells were fixed by a cold solution of ethanol (95%) and acetic acid (5%) for 5 min at −20° C. Cell membranes were permeabilized and non-specific binding sites were blocked with a solution of PBS containing 0.1% of saponin and 1% FCS for 15 min at room temperature. Cells were incubated for 2 hours with the following primary antibodies:
a) a mouse monoclonal antibody anti microtubule-associated-protein 2 (MAP-2) at dilution of 1/400 in PBS containing 1% fetal calf serum and 0.1% of saponin.
b) a rabbit polyclonal antibody anti-nuclear TAR DNA-binding protein 43 (TDP-43) at dilution of 1/100 in PBS containing 1% fetal calf serum and 0.1% of saponin.

These antibodies were revealed with an Alexa Fluor 488 goat anti-mouse IgG at the dilution 1/400 and a goat anti-rabbit IgG coupled with an Alexa Fluor 568 at the dilution 1/400, in PBS containing 1% FCS, 0.1% saponin, for 1 hour at room temperature. Nuclei were counterstained with the fluorescent dye Hoechst (sigma 1/1000), a marker of total cell survival.

### Automatic Computer Analysis

For each condition, 30 pictures (representative of the all well area) per well were automatically taken using ImageXpress® (Molecular Devices) with 20× magnification, using the same acquisition parameters. From images, analyses were directly and automatically performed by MetaXpress® (Molecular Devices). The following endpoints will be automatically assessed:
Analysis of neuron survival (MAP-2 staining, number of neurons),
Analysis of neurite network (MAP-2 staining, total neurite length in μm),
Analysis of cytoplasmic TDP-43 in MAP-2 positive neurons (overlapping between MAP-2 and cytoplasmic TDP-43 in μm²).

### Statistical Analysis

All values are expressed as mean+/−SEM (standard error of the mean). Statistical analysis was performed using one-way ANOVA, followed by a Dunnett's or a Fisher's LSD test. p<0.05 will be considered significant.

### Results

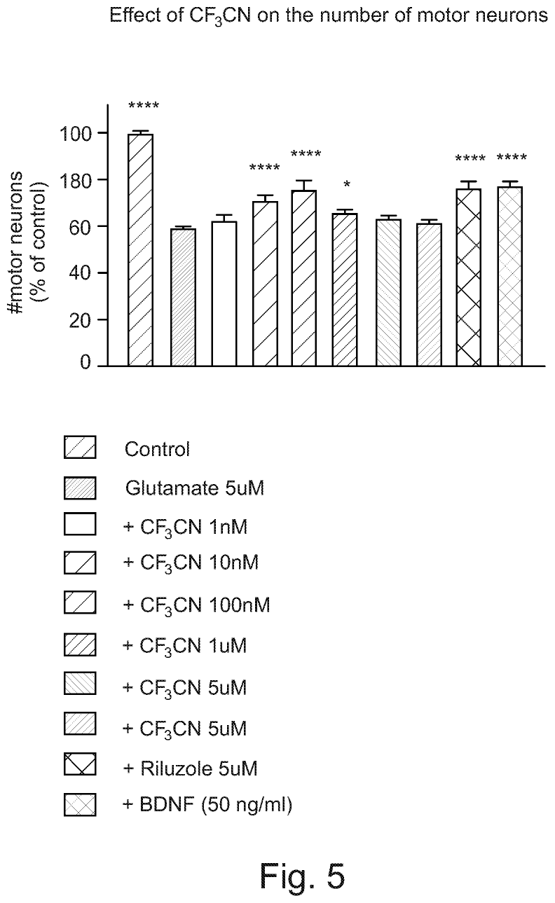

FIG. 5 details the effect of CF₃CN on the number of motor neurons. As can be seen both riluzole and BDNF produced a statistically significant increase in the number of motor neurons compared to the control. In addition, CF₃CN at concentrations of 10 nm, 100 nm and 1 μm also produced a statistically significant increase in the number of motor neurons compared to the control.

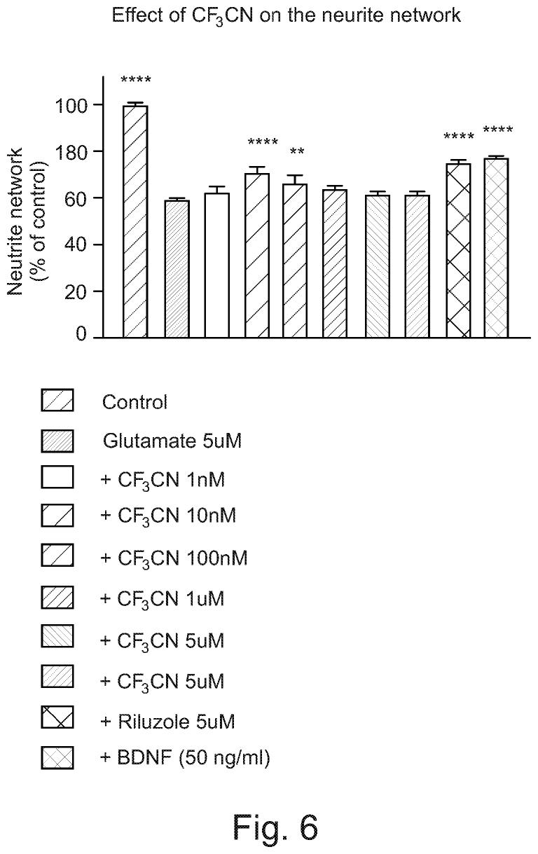

FIG. 6 details the effect of CF₃CN on the neurite network. As detailed both the positive controls increased the neurite network in a statistically significant manner compared to the control. In addition, CF₃CN at 10 and 100 nm was shown to increased the neurite network in a statistically significant manner compared to the control.

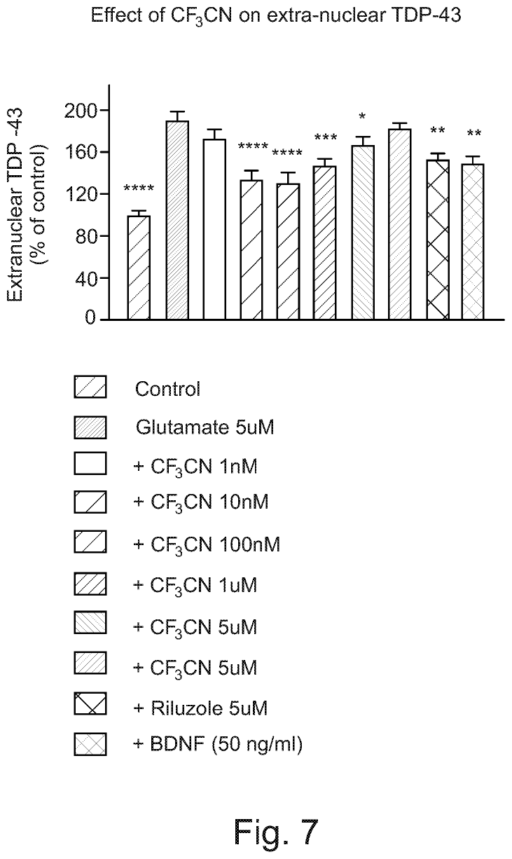

FIG. 7 details the effect of CF₃CN on number of extranuclear TDP-43. Both of the positive controls decreased the number of extranuclear TDP-43 in a statistically significant manner compared to the control. Furthermore, CF₃CN at all concentrations except the highest and the lowest was found to decreased the number of extranuclear TDP-43 in a statistically significant manner compared to the control.

Surprisingly, CF₃CN at 10 nm and 100 nm was shown to have a greater decrease in the level of extranuclear TDP-43 than the positive controls.

### Conclusion

The data detailed in this Example shows that the test compound CF₃CN is able to have a positive effect in an in vitro model of ALS, in some cases it was shown to behave better than the positive control and the standard of care for ALS riluzole.

As such this compound may be beneficial in the treatment of diseases and conditions associated with movement disorders.

1. CF₃CN for use in the treatment of diseases or conditions associated with movement disorder.

2. CF₃CN for use according to claim 1, wherein the neurodegenerative diseases or conditions is selected from the group: ataxia; cerebral palsy; cervical dystonia; chorea; dystonia; epilepsy; functional movement disorder; Huntington's disease; multiple sclerosis; multiple system atrophy; myoclonus; Parkinson's disease; Parkinsonism; progressive supranuclear palsy; restless legs syndrome; tardive dyskinesia; Tourette syndrome; tremor disorders such as essential tremor; and Wilson's disease.

3. CF₃CN for use according to claim 2, wherein the neurodegenerative diseases or conditions is treatment resistant.

4. CF₃CN for use according to claim 1, wherein the neurodegenerative diseases or conditions is a drug-induced movement disorder.

5. CF₃CN for use according to claim 4, wherein the drug-induced movement disorder is akathisia; tremor; serotonin syndrome; acute dystonic reaction; neuroleptic malignant syndrome; Parkinsonism; or tardive drug-induced movement disorder.

6. CF₃CN for use according to claim 1, wherein the disease or condition associated with movement disorder is additionally associated with cognitive impairment.

7. CF₃CN for use according to claim 6, wherein the disease or condition is taken from the group consisting of: Huntington's disease; Parkinson's disease; and essential tremor.

8. CF₃CN for use according to claim 1, wherein the CF₃CN is administered with one or more pharmaceutically acceptable excipients.

9. CF₃CN for use according to claim 1, wherein the CF₃CN is formulated in a dosage form selected from a liquid, a lozenge, a fast-disintegrating tablet, a lyophilized preparation, a film, a spray, an aerosol, a sustained-release tablet or capsule, a modified release, a sustained relief, a tablet, a capsule a cream, an ointment, or a mucoadhesive.

10. CF₃CN for use according to claim 1, wherein the CF₃CN is administered as a single daily dose.

11. CF₃CN for use according to claim 1, wherein the CF₃CN is administered as multiple daily doses.

12. CF₃CN for use according to claim 11, wherein the CF₃CN is administered two, three, four or five times per day.

13. CF₃CN for use according to claims 10 to 12, wherein each dose comprises at least 0.001 mg of CF₃CN.

14. CF₃CN for use according to claims 10 to 12, wherein each dose comprises between about 0.001 mg and about 500 mg of CF₃CN.

15. CF₃CN for use according to claims 10 to 12, wherein each dose comprises between about 500 mg and about 1000 mg of CF₃CN.

16. CF₃CN for use according to claim 1, wherein the CF₃CN is administered with one or more additional drug products.

17. A method of treating diseases or conditions associated with movement disorder in a subject in need thereof, the method comprising administering to the subject a therapeutically effective amount of CF₃CN.
